(19) United States
(12) Patent Application Publication
(10) Pub. No.: US 2008/0089897 A1
(43) Pub. Date: Apr. 17, 2008

(54) ActRIIB Fusion Polypeptides and Uses Therefor

(75) Inventor: Neil M. Wolfman, Dover, MA (US)

Correspondence Address: WYETH/FINNEGAN HENDERSON, LLP, 901 NEW YORK AVENUE, NW, WASHINGTON, DC, 20001-4413, US

(73) Assignee: Wyeth, Madison, NJ (US)

(21) Appl. No.: 11/835,248

(22) Filed: Aug. 7, 2007

Related U.S. Application Data
(63) Continuation of application No. 10/689,677, filed on Oct. 22, 2003.
(60) Provisional application No. 60/421,041, filed on Oct. 25, 2002.

(51) Int. Cl. A61K 39/395; A61P 21/00; A61P 25/00; A61P 3/00

(52) U.S. Cl. 424/178.1

(57) ABSTRACT

Methods and compositions for inhibiting growth and differentiation factor-8 (GDF-8) activity in vitro and in vivo are provided. The methods and composition can be used for diagnosing, preventing, or treating degenerative disorders of muscle, bone, or glucose homeostasis.

This application is a continuation of U.S. patent application Ser. No. 10/689,677, filed Oct. 22, 2003, which claims priority to U.S. provisional patent application No. 60/421,041, filed on Oct. 25, 2002, both of which are incorporated herein by reference in their entirety.

### TECHNICAL FIELD

This technical field relates to inhibitors of growth and differentiation factor-8 (GDF-8), including soluble forms of activin type II receptors, and fragments thereof, especially those that inhibit GDF-8 activity in vivo. The field further relates to methods for diagnosing, preventing, or treating degenerative disorders of muscle, bone, or glucose homeostasis.

### BACKGROUND

The TGF-β family is a number of structurally-related growth factors, all of which possess physiologically important growth-regulatory and morphogenetic properties (Kingsley et al. (1994) Genes Dev., 8:133-146; Hoodless et al. (1998) Curr. Topics Microbiol. Immunol., 228:235-272). These factors include bone morphogenetic proteins (BMP), activin, inhibin, mullerian inhibiting substance, glial-derived neurotrophic factor, and a still growing number of growth and differentiation factors (GDF), such as GDF-8. Many of these proteins are highly homologous. For example, human BMP-11, also known as GDF-11, is 90% identical to GDF-8 at the amino-acid level (Gamer et al. (1999) Dev. Biol. 208:222-232; Nakashima et al. (1999) Mech. Dev. 80:185-189).

Most members of the TGF-β family are known to transduce their signals through the formation of heteromeric complexes of two different types of serine/threonine kinase receptors expressed on the cell surface, i.e., type I receptors of about 50-55 kDa and type II receptors of more than 70 kDa. Type I receptors do not bind ligands directly; rather, they participate in signal transduction by associating with the type II receptors, which do bind ligand molecules. The TGF-β system is highly conserved throughout the animal kingdom. (For a review of the TGF-β system, see Massague (2000) Nature Rev. Mol. Cell Biol 1:16-178; and Moustakas et al. (2001) J. Cell Sci. 114:4359-4369)

Activin type II receptor has been previously described in U.S. Pat. No. 5,885,794. Activin was originally purified from ovarian follicular fluid as a protein that has a stimulatory effect on production of follicle-stimulating hormone in the pituitary gland. Five isoforms of activin type II receptor have been identified in activin-responsive cells. Based on in vitro studies, these receptors may be shared by members of the TGF-β family (Attisano et al. (1996) Mol. Cell. Biol. 16:1066-1073). The present invention is based, in part, on the discovery that the type II activin receptor, termed ActRIIB, can bind to growth and differentiation factor-8 (GDF-8) in addition to activin.

GDF-8 is involved in the regulation of critical biological processes in the skeletal muscle and osteogenesis. GDF-8 is highly expressed in the developing and adult skeletal muscle. GDF-8 knockout transgenic mice are characterized by a marked hypertrophy and hyperplasia of the skeletal muscle (McPherron et al. (1997) Nature 387:83-90) and altered cortical bone structure (Hamrick et al. (2000) Bone 27 (3):343-349). Similar increases in skeletal muscle mass are evident in naturally occurring mutations of GDF-8 in cattle (Ashmore et al. (1974) Growth 38:501-507; Swatland et al. (1994) J. Anim. Sci. 38:752-757; McPherron et al. (1997) Proc. Natl. Acad. Sci. U.S.A. 94:12457-12461; and Kambadur et al. (1997) Genome Res. 7:910-915). Studies have indicated that muscle wasting associated with HIV-infection is accompanied by an increase in GDF-8 expression (Gonzalez-Cadavid et al. (1998) Proc. Natl. Acad. Sci. U.S.A. 95:14938-14943). GDF-8 has also been implicated in the production of muscle-specific enzymes (e.g., creatine kinase) and proliferation of myoblast cells (WO 00/43781). In addition to its growth-regulatory and morphogenetic properties, GDF-8 may also be involved in a number of other physiological processes, including glucose homeostasis in the development of type 2 diabetes, impaired glucose tolerance, metabolic syndromes (e.g., syndrome X), insulin resistance induced by trauma such as burns or nitrogen imbalance, and adipose tissue disorders, such as obesity (Kim et al. (2001) BBRC 281:902-906).

A number of human and animal disorders are associated with functionally impaired muscle tissue, e.g., muscular dystrophy including Duchenne's muscular dystrophy), amyotrophic lateral sclerosis (ALS), muscle atrophy, organ atrophy, frailty, congestive obstructive pulmonary disease, sarcopenia, cachexia, and muscle wasting syndrome caused by other diseases and conditions. To date, very few reliable or effective therapies have been developed to treat these disorders.

There are also a number of conditions associated with a loss of bone, which include osteoporosis and osteoarthritis, especially in the elderly and/or postmenopausal women. In addition, metabolic bone diseases and disorders include low bone mass due to chronic glucocorticoid therapy, premature gonadal failure, androgen suppression, vitamin D deficiency, secondary hyperparathyroidism, nutritional deficiencies, and anorexia nervosa. Currently available therapies for these conditions work by inhibiting bone resorption. A therapy that promotes new bone formation would be a desirable alternative to these therapies.

Thus, a need exists to develop new therapies that contribute to an overall increase of muscle mass and/or bone density, especially, in humans. It is an object of the present invention to provide safe and effective therapeutic methods for muscle and/or bone-associated disorders. It is another object of the invention to provide methods of increasing muscle mass and/or bone density in mammals. It is yet another object of the invention to provide inhibitors of GDF-8 that are safe and effective in vivo.

Still another object of the invention is to provide soluble forms of activin type II receptor ActRIIB and/or functional fragments thereof that are stable in vivo and bind GDF-8 with high specificity and affinity.

Additional objects of the invention will be set forth in part in the description which follows, and in part will be obvious from the description, or may be learned by practice of the invention. Various objects, aspects, and advantages of the invention will be realized and attained by means of the elements and combinations particularly pointed out in the appended claims.

### SUMMARY

Methods for treating muscle and bone degenerative disorders are provided herein. The methods are also useful for increasing muscle mass and bone density in normal animals.

Also provided are methods for inhibiting GDF-8 activity associated with negative regulation of skeletal muscle mass and bone density.

Stabilized soluble ActRIIB forms and fragments thereof that bind and inhibit GDF-8 in vitro and in vivo are provided. The presently disclosed soluble ActRIIB forms possess pharmacokinetic properties that make them suitable as therapeutic agents.

Other aspects provide compositions containing the presently described ActRIIB fusion polypeptides and their use in methods of inhibiting or neutralizing GDF-8, including methods of treatment of the human or animals. The disclosed ActRIIB fusion polypeptides may be used to treat or prevent conditions in which an increase in muscle tissue or bone density is desirable. For example, the ActRIIB fusion polypeptides may also be used in therapies to repair damaged muscle, e.g., myocardium, diaphragm, etc. Exemplary disease and disorders include muscle and neuromuscular disorders such as muscular dystrophy including Duchenne's muscular dystrophy); amyotrophic lateral sclerosis; muscle atrophy; organ atrophy; frailty; carpal tunnel syndrome; congestive obstructive pulmonary disease; sarcopenia, cachexia and other muscle wasting syndromes; adipose tissue disorders such as obesity; type 2 diabetes; impaired glucose tolerance; metabolic syndromes (e.g., syndrome X); insulin resistance induced by trauma such as burns or nitrogen imbalance; and bone degenerative disease such as osteoarthritis and osteoporosis.

The modified ActRIIB forms utilized in the methods of the invention are ActRIIB fusion polypeptides comprising (a) a first amino acid sequence derived from the ActRIIB extracellular domain and (b) a second amino acid sequence derived from the constant region of an antibody.

In certain embodiments, the first sequence comprises all or a portion of an extracellular domain of human ActRIIB, or is a mutation of such a sequence. The second sequence may be derived from the Fc portion of an antibody, or is a mutation of such a sequence.

In further embodiments, the second sequence is linked to the C-terminus or the N-terminus of the first amino acid sequence, with or without being linked by a linker sequence.

Therapeutic methods for treating muscle and/or bone degenerative disorders are also provided. Exemplary disease and disorders include muscle and neuromuscular disorders (such as muscular dystrophy), muscle atrophy, congestive obstructive pulmonary disease, muscle wasting syndrome, sarcopenia, cachexia, adipose tissue disorders such as obesity, type 2 diabetes, impaired glucose tolerance, metabolic syndrome (e.g., syndrome X), insulin resistance induced by trauma (e.g., burns), and bone degenerative disease such as osteoporosis.

In addition, the presently disclosed ActRIIB fusion polypeptides may be used as a diagnostic tool to quantitatively or qualitatively detect GDF-8 or fragments thereof in a biological sample. The presence or amount of GDF-8 detected can be correlated with one or more of the medical conditions listed above.

An isolated nucleic acid encoding an ActRIIB fusion polypeptide used in the methods of the invention is also provided. Further provided are expression vectors comprising the nucleic acid; host cells comprising the expression vectors; and methods for producing the nucleic acid.

Yet another aspect provides a method for identifying therapeutic agents useful in treatment of muscle and bone disorders.

It is to be understood that both the foregoing general description and the following detailed description are exemplary and explanatory only and are not restrictive of the invention, as claimed.

### BRIEF DESCRIPTION OF THE FIGURES

FIG. 1 shows binding of biotinylated GDF-8 and BMP-11 to ActRIIB-Fc.

FIG. 2 shows results of reporter gene assays in which ActRIIB-Fc has been tested.

FIG. 3 depicts results of a pharmacokinetic study in which C57B6/SCID mice utilizing a single intravenous (IV) or intraperitoneal (IP) administration of ActRIIB-Fc.

### BRIEF DESCRIPTION OF THE SEQUENCES

The following table is provided as a reference for the sequences referred to in this application.

| Reference | Type* | Sequence |
|---|---|---|
| SEQ ID NO: 1 | AA | ActRIIB |
| SEQ ID NO: 2 | AA | GDF-8 |
| SEQ ID NO: 3 | AA | ActRIIB-Fc |
| SEQ ID NO: 4 | DNA | Encodes SEQ ID NO: 3 |
| SEQ ID NO: 5 | AA | Linker |
| SEQ ID NO: 6 | AA | Enterokinase cleavage site |

*AA = amino acid

### DETAILED DESCRIPTION

### I. Definitions

The term “ActRIIB” refers to any isoform of activin type II receptor or a fragment thereof that is capable of specifically binding GDF-8. The term is not limited to any particular species of origin, method of production, and other characteristics of ActRIIB. The term includes recombinantly produced ActRIIB or its fragments, and particularly, the GDF-8 binding domain of human ActRIIB. The term also encompasses allelic and splice variants of ActRIIB, their homologues, and orthologues and sequences thereof containing introduced mutations (substitutions, additions, or deletions), e.g., those introduced by recombinant techniques.

The term “degenerative disorder of muscle, bone, or glucose homeostasis” refers to a number of disorders and diseases associated with GDF-8 and/or other members of the TGF-β superfamily, e.g., BMP-11. Example of such disorders include, but are not limited to, metabolic disorders such as type 2 diabetes, impaired glucose tolerance, metabolic syndrome (e.g., syndrome X), and insulin resistance induced by trauma (e.g., burns or nitrogen imbalance); adipose tissue disorders (e.g., obesity); muscle and neuromuscular disorders such as muscular dystrophy (including Duchenne's muscular dystrophy); amyotrophic lateral sclerosis (ALS); muscle atrophy; organ atrophy; frailty; carpal tunnel syndrome; congestive obstructive pulmonary disease; and sarcopenia, cachexia and other muscle wasting syndromes. Other examples include osteoporosis, especially in the elderly and/or postmenopausal women; glucocorticoid-induced osteoporosis; osteopenia; osteoarthritis; and osteoporosis-related fractures. Yet further examples include low bone mass due to chronic glucocorticoid therapy, premature gonadal failure, androgen suppression, vitamin D deficiency, secondary hyperparathyroidism, nutritional deficiencies, and anorexia nervosa.

The term “effective amount” refers to that amount of the compound which results in amelioration of symptoms in a patient or a desired biological outcome (e.g., increasing skeletal muscle mass and/or bone density). Such amount should be sufficient to reduce the activity of GDF-8 associated with negative regulation of skeletal muscle mass and bone density. The effective amount can be determined as described in the subsequent sections.

The term “GDF-8 binding domain,” when used in relation to ActRIIB, refers to the extracellular domain of ActRIIB or a part thereof necessary for binding to GDF-8, i.e., a portion of the ActRIIB extracellular domain responsible for specific binding to GDF-8.

The term “TGF-β superfamily” refers to a family of structurally related growth factors. This family of related growth factors is well known in the art (Kingsley et al. (1994) Genes Dev. 8:133-146; Hoodiess et al. (1998) Curr. Topics Microbiol. Immunol. 228:235-72). The TGF-β superfamily includes bone morphogenetic proteins (BMP), activin, inhibin, mullerian inhibiting substance, glial-derived neurotrophic factor, and a still growing number of growth and differentiation factors (GDF), such as GDF-8 (myostatin). Many of such proteins are structurally and/or functionally related to GDF-8. For example, human BMP-11, also known as GDF-11, is 90% identical to GDF-8 at the amino-acid level (Gamer et al. (1999) Dev. Biol. 208:222-232; Nakashima et al. (1999) Mech. Dev. 80:185-189).

The term “GDF-8” refers to a specific growth and differentiation factor-8 and, where appropriate, should be understood to include any factor that is structurally or functionally related to GDF-8 such as BMP-11 and other factors that belong to the TGF-β superfamily. The term refers to the full-length unprocessed precursor form of GDF-8, as well as the mature and propeptide polypeptides resulting from post-translational cleavage. The term also refers to any fragments and variants of GDF-8 that retain one or more biological activities associated with GDF-8 as discussed herein. The amino acid sequence of mature human GDF-8 is provided in SEQ ID NO:2. The present invention relates to GDF-8 from all vertebrate species, including, but not limited to, human, bovine, chicken, murine, rat, porcine, ovine, turkey, baboon, and fish (for sequence information, see, e.g., McPherron et al. (1997) Proc. Natl. Acad. Sci. U.S.A. 94:12457-12461).

The term “mature GDF-8” refers to the protein that is cleaved from the carboxy-terminal domain of the GDF-8 precursor protein. The mature GDF-8 may be present as a monomer, homodimer, or in a GDF-8 latent complex. Depending on conditions, mature GDF-8 may establish equilibrium between any or all of these different polypeptides. In its biologically active form, the mature GDF-8 is also referred to as “active GDF-8.”

The term “GDF-8 propeptide” refers to the polypeptide that is cleaved from the amino-terminal domain of the GDF-8 precursor protein. The GDF-8 propeptide is capable of binding to the propeptide binding domain on the mature GDF-8.

The term “GDF-8 latent complex” refers to the complex of proteins formed between the mature GDF-8 homodimer and the GDF-8 propeptide. It is believed that two GDF-8 propeptides associate with the two molecules of mature GDF-8 in the homodimer to form an inactive tetrameric complex. The latent complex may include other GDF-8 inhibitors in place of or in addition to one or both of the GDF-8 propeptides.

The term “GDF-8 activity” refers to one or more of physiologically growth-regulatory or morphogenetic activities associated with active GDF-8 protein. For example, active GDF-8 is a negative regulator of skeletal muscle. Active GDF-8 can also modulate the production of muscle-specific enzymes (e.g., creatine kinase), stimulate myoblast proliferation, and modulate preadipocyte differentiation to adipocytes. Procedures for assessing GDF-8 activity in vivo and in vitro include, but are not limited to, for example, reporter gene assays (see Example 6) or in vivo tests involving measurements of muscle and/or bone parameters (see Examples 8, 9, and 10).

The term “Fc portion” refers to the C-terminal fragment of an immunoglobulin generated by proteolysis with papain, or a functional equivalent derived therefrom. The term “Fc portion” should be understood to encompass recombinantly produced Fc fragments, including those derived from any antibody isotype, e.g., IgG, IgA, IgE, IgM, and any of the isotype subclasses. The term “constant region of an antibody” refers to a C-terminal portion of an immunoglobulin, comprising the Fc portion and adjacent sequences so long as these sequences do not include variable regions of the antibody, such as complementarity determining regions (CDRs). The constant region of an antibody is the same in all antibodies of a particular isotype.

As used herein, the term “hybridization under stringent conditions” is intended to describe conditions for hybridization and washes under which nucleotide sequences that are significantly identical or homologous to each other remain complementarily bound to each other. The conditions are such that sequences at least about 70%, more preferably at least about 80%, even more preferably at least about 85-90% identical remain bound to each other. The percent identity is determined as described in Altschul et al. (1997) Nucleic Acids Res. 25:3389-3402.

Stringent conditions are known in the art and can be found in Current Protocols in Molecular Biology, John Wiley & Sons, Inc. (eds. Ausubel et al. 1995), sections 2, 4, and 6. Additionally, stringent conditions are described in Sambrook et al. (1989) Molecular Cloning: A Laboratory Manual, 2nd ed. Cold Spring Harbor Press, chapters 7, 9, and 11. An example of stringent hybridization conditions is hybridization in 4× sodium chloride/sodium citrate (SSC) at about 65-70° C. or hybridization in 4×SSC plus 50% formamide at about 42-50° C., followed by one or more washes in 1×SSC, at about 65-70° C. When using nylon membranes, for instance, an additional non-limiting example of stringent hybridization conditions is hybridization in 0.25-0.5 M NaH₂PO₄, 7% SDS at about 65° C., followed by one or more washes at 0.02 M NaH₂PO₄, 1% SDS at 65° C. See, e.g., Church et al. (1984) Proc. Natl. Acad. Sci. U.S.A. 81:1991-1995. It will be understood that additional reagents may be added to hybridization and/or wash buffers, e.g., blocking agents (BSA or salmon sperm DNA), detergents (SDS), chelating agents (EDTA), Ficoll, PVP, etc.

The term “inhibitor,” when used in relationship to GDF-8 or its activity, includes any agent capable of inhibiting activity, expression, processing, or secretion of GDF-8. Such inhibitors include proteins, antibodies, peptides, peptidomimetics, ribozymes, anti-sense oligonucleotides, double-stranded RNA, and other small molecules, which inhibit GDF-8. Such inhibitors are said to “inhibit,” “neutralize,” or “reduce” the biological activity of GDF-8 protein.

The terms “neutralize,” “neutralizing,” “inhibitory,” and their cognates refer to a reduction in the activity of GDF-8 by a GDF-8 inhibitor, relative to the activity of GDF-8 in the absence of the same inhibitor. The reduction in activity is preferably at least about 10%, 20%, 30%, 40%, 50%, 60%, 70%, 80%, 90%, or higher.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition or at least 70% to 80% (w/w) pure, at least 80%-90% pure, 90-95% pure; or at least 95%, 96%, 97%, 98%, 99%, or 100% pure.

The term “mammal” refers to any animal classified as such, including humans, domestic and farm animals, zoo, sports, or pet animals, such as dogs, horses, cats, sheep, pigs, cows, etc.

The term “specific interaction,” or “specifically binds,” or the like, means that two molecules form a complex that is relatively stable under physiologic conditions. The term is also applicable where, e.g., an antigen-binding domain is specific for a particular epitope, which is carried by a number of antigens, in which case the antibody carrying the antigen-binding domain will be able to bind to the various antigens carrying the epitope. Thus, an antibody may specifically bind, for example, BMP-11 and GDF-8 as long as it binds to the epitope, which is carried by both.

Specific binding is characterized by a high affinity and a low to moderate capacity. Nonspecific binding usually has a low affinity with a moderate to high capacity. Typically, the binding is considered specific when the affinity constant Kₐ is higher than 10⁶ M⁻¹, or preferably higher than 10⁸ M⁻¹. If necessary, nonspecific binding can be reduced without substantially affecting specific binding by varying the binding conditions. Such conditions are known in the art, and a skilled artisan using routine techniques can select appropriate conditions. The conditions are usually defined in terms of concentration of the ActRIIB fusion polypeptide, ionic strength of the solution, temperature, time allowed for binding, concentration of non-related molecules (e.g., serum albumin, milk casein), etc. Exemplary conditions are set forth in Examples 5 and 6.

The phrase “substantially as set out” means that a relevant amino acid sequence is at least 70%, 75%, 80%, 85%, 90%, 95%, 97%, 98%, or 99% identical to a given sequence. By way of example, such sequences may be variants derived from various species, or they may be derived from the given sequence by truncation, deletion, amino acid substitution or addition. Percent identity between two amino acid sequences is determined by standard alignment algorithms such as, for example, Basic Local Alignment Tool (BLAST) described in Altschul et al. (1990) J. Mol. Biol. 215:403-410, the algorithm of Needleman et al. (1970) J. Mol. Biol. 48:444-453, or the algorithm of Meyers et al. (1988) Comput. Appl. Biosci. 4:11-17.

The term “treatment” refers to both therapeutic treatment and prophylactic/preventative treatment. Those in need of treatment may include individuals already having a particular medical disorder as well as those who may ultimately acquire the disorder (i.e., those needing preventative measures, such as, for example, post-menopausal women with a family history of osteoporosis, or obese patients with a family history of type 2 diabetes or somewhat elevated blood sugar readings).

### II. ActRIIB Fusion Polypeptides

The present invention provides modified activin type II receptor ActRIIB that binds GDF-8 and inhibits its activity in vitro and/or in vivo. In particular, the presently disclosed ActRIIB fusion polypeptides inhibit the GDF-8 activity associated with negative regulation of skeletal muscle mass and bone density. The ActRIIB fusion polypeptides of the invention are soluble and possess pharmacokinetic properties that make them suitable for therapeutic use, e.g., extended circulatory half-life and/or improved protection from proteolytic degradation.

The ActRIIB fusion polypeptides of the invention comprise (a) a first amino acid sequence derived from the extracellular domain of ActRIIB and (b) a second amino acid sequence derived from the constant region of an antibody. The full amino acid and DNA sequences of a particular illustrative embodiment of the ActRIIB fusion protein are set forth in SEQ ID NO:3 and SEQ ID NO:4, respectively.

The first amino acid sequence is derived from all or a portion of the ActRIIB extracellular domain and is capable of binding GDF-8 specifically. In some embodiments, such a portion of the ActRIIB extracellular domain may also bind BMP-11 and/or activin, or other growth factors. In certain embodiments, the first amino acid sequence is identical to or is substantially as set out in SEQ ID NO:3 from about amino acid (aa) 23 to about aa 138 or from about aa 19 to about aa 134 in SEQ ID NO:1. The difference between SEQ ID NO:1 and SEQ ID NO:3 is that aa 64 of SEQ ID NO:1 is Ala, whereas the corresponding aa 68 in SEQ ID NO:3 is Arg. Additionally, other variances in the sequence of ActRIIB are possible, for example, aa 16 and aa 17 in SEQ ID NO:1 can be substituted with Cys and Ala, respectively. In some other embodiments, the first amino acid sequence comprises at least 20, 30, 40, 50, 60, 70, 80, 90, 100, 110, or 120 contiguous amino acids from about aa 23 and about aa 138 of SEQ ID NO:3 or about aa 19 and about aa 134 of SEQ ID NO:1. Such a sequence can be truncated so long as the truncated sequence is capable of specifically binding GDF-8. Binding to GDF-8 can be assayed using methods known in the art or as described in Examples 5 and 6.

The second amino acid sequence is derived from the constant region of an antibody, particularly the Fc portion, or is a mutation of such a sequence. In some embodiments, the second amino acid sequence is derived from the Fc portion of an IgG. In related embodiments, the Fc portion is derived from IgG that is IgG₁, IgG₄, or another IgG isotype. In a particular embodiment, the second amino acid sequence comprises the Fc portion of human IgG₁ as set forth in SEQ ID NO:3 amino acids 148 to 378), wherein the Fc portion of human IgG₁ has been modified to minimize the effector function of the Fc portion. Such modifications include changing specific amino acid residues which might alter an effector function such as Fc receptor binding (Lund et al. (1991) J. Immun. 147:2657-2662 and Morgan et al. (1995) Immunology 86:319-324), or changing the species from which the constant region is derived. Antibodies may have mutations in the C_{H}2 region of the heavy chain that reduce effector function, i.e., Fc receptor binding and complement activation. For example, antibodies may have mutations such as those described in U.S. Pat. Nos. 5,524,821 and 5,648,260. In the IgG₁ or IgG₂ heavy chain, for example, such mutations may be made at amino acid residues corresponding to amino acids 234 and 237 in the full-length sequence of IgG₁ or IgG₂. Antibodies may also have mutations that stabilize the disulfide bond between the two heavy chains of an immunoglobulin, such as mutations in the hinge region of IgG₄, as disclosed in Angal et al. (1993) Mol. Immunol. 30:105-108.

In certain embodiments, the second amino acid sequence is linked to the C-terminus or the N-terminus of the first amino acid sequence, with or without being linked by a linker sequence. The exact length and sequence of the linker and its orientation relative to the linked sequences may vary. The linker may be, for example, (Gly-Ser)₂ SEQ ID NO:5). The linker may comprise 2, 10, 20, 30, or more amino acids and is selected based on properties desired such as solubility, length and steric separation, immogenicity, etc. In certain embodiments, the linker may comprise a sequence of a proteolytic cleavage site, such as the enterokinase cleavage site Asp-Asp-Asp-Lys (SEQ ID NO:6), or other functional sequences useful, for example, for purification, detection, or modification of the fusion protein.

It will be understood by one of ordinary skill in the art that certain amino acids in a sequence of any protein may be substituted for other amino acids without adversely affecting the activity of the protein. It is thus contemplated that various changes may be made in the amino acid sequences the sequence of the ActRIIB fusion polypeptides of the invention, or DNA sequences encoding such polypeptides, without appreciable loss of their biological activity or utility. The biological activity of ActRIIB can be measured as described in Examples 6-10. Such changes may include, but are not limited to, deletions, insertions, truncations, and substitutions.

In certain embodiments, additional fusions of any of ActRIIB fusion polypeptides of the invention to amino acid sequences derived from other proteins may be constructed. Desirable fusion sequences may be derived from proteins having biological activity different from that of ActRIIB, for example, cytokines, growth and differentiation factors, enzymes, hormones, other receptor components, etc. Also, ActRIIB fusion polypeptides may be chemically coupled, or conjugated, to other proteins and pharmaceutical agents. Such modification may be designed to alter the pharmacokinetics and/or biodistribution of the resultant composition.

The ActRIIB fusion polypeptides of the invention can be glycosylated, pegylated, or linked to another nonproteinaceous polymer. For instance, the presently disclosed ActRIIB fusion polypeptides may be linked to one of a variety of nonproteinaceous polymers, e.g., polyethylene glycol, polypropylene glycol, or polyoxyalkylenes, in the manner set forth in U.S. Pat. No. 4,640,835; U.S. Pat. No. 4,496,689; U.S. Pat. No. 4,301,144; U.S. Pat. No. 4,670,417; U.S. Pat. No. 4,791,192; or U.S. Pat. No. 4,179,337. The ActRIIB fusion polypeptides are chemically modified by covalent conjugation to a polymer to increase their circulating half-life, for example. Exemplary polymers, and methods to attach them to peptides, are also shown in U.S. Pat. Nos. 4,766,106; 4,179,337; 4,495,285; and 4,609,546.

The ActRIIB fusion polypeptides of the invention may be modified to have an altered glycosylation pattern (i.e., altered from the original or native glycosylation pattern). As used herein, “altered” means having one or more carbohydrate moieties deleted, and/or having one or more glycosylation sites added to the original sequence. Addition of glycosylation sites to the presently disclosed modified ActRIIB may be accomplished by altering the amino acid sequence to contain glycosylation site consensus sequences well known in the art. Another means of increasing the number of carbohydrate moieties is by chemical or enzymatic coupling of glycosides to the amino acid residues. These methods are described in WO 87/05330, and in Aplin et al. (1981) Crit. Rev. Biochem. 22:259-306. Removal of any carbohydrate moieties present on ActRIIB may be accomplished chemically or enzymatically as described by Hakimuddin et al. (1987) Arch. Biochem. Biophys. 259:52; Edge et al. (1981) Anal. Biochem. 118:131 and by Thotakura et al. (1987) Meth. Enzymol. 138:350.

The ActRIIB fusion polypeptides of the invention may also be tagged with a detectable or functional label. Detectable labels include radiolabels such as ¹³¹I or ⁹⁹Tc, which may be attached to ActRIIB fusion polypeptides of the invention using conventional chemistry known in the art. Labels also include enzyme labels such as horseradish peroxidase or alkaline phosphatase. Labels further include chemical moieties such as biotin, which may be detected via binding to a specific cognate detectable moiety, e.g., labeled avidin.

One of skill in the art will recognize that the ActRIIB fusion polypeptides of the invention may be used to detect, measure, and inhibit proteins other than GDF-8, BMP-11, and activin. Nonlimiting examples of such proteins, for example, sequences of GDF-8 derived from various species (orthologues), are described in the present specification.

### III. Nucleic Acids, Cloning and Expression Systems

The present disclosure provides an isolated nucleic acid encoding a soluble ActRIIB that can be utilized in the methods of the present invention. The nucleic acid of the invention comprises a coding sequence for at least one ActRIIB fusion polypeptide of the invention as described herein. In certain embodiments, the nucleic acid comprises the sequence, or is derived from the sequence set forth in SEQ ID NO:4. In certain other embodiments, the nucleic acid sequence such that it encodes amino acids sequences from about aa 23 and about aa 138 of SEQ ID NO:3 or from about aa 19 and about aa 134 of SEQ ID NO:1.

The disclosure also provides constructs in the form of plasmids, vectors, transcription or expression cassettes which comprise at least one nucleic acid of the invention as above.

The disclosure also provides a host cell, which comprises one or more constructs as above. A nucleic acid encoding any one of the ActRIIB fusion polypeptides, as provided, is itself an aspect of the present invention, as is a method of production of the encoded product. Production of the encoded ActRIIB fusion polypeptides may be achieved by expression recombinant host cells containing the nucleic acid under appropriate culturing conditions. Following expression, an ActRIIB fusion polypeptide is isolated and/or purified using any suitable technique, then used as appropriate. Exemplary procedures for expression and purification are presented in Examples 3 and 4.

Specific ActRIIB fusion polypeptides and encoding nucleic acid molecules and vectors according to the present invention may be obtained, isolated and/or purified, e.g., from their natural environment, in substantially pure or homogeneous form, or in the case of nucleic acid, free or substantially free of nucleic acid or genes origin other than the sequence encoding a polypeptide with the required function. Nucleic acids, according to the present invention, may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

The invention also encompasses sequences that are at least 100, 200, 300, 400, 500, 600, 700, 800, 900, or 1000 nucleotides long and hybridize under stringent hybridization conditions to the nucleic acid set forth in SEQ ID NO:4.

Systems for cloning and expression of a polypeptide in a variety of different host cells are well known. Suitable host cells include bacteria, mammalian cells, and yeast and baculovirus systems. Mammalian cell lines available in the art for expression of a heterologous polypeptide include Chinese hamster ovary cells, HeLa cells, baby hamster kidney cells, NS0 mouse melanoma cells and many others. A common bacterial host is E. coli. For other cells suitable for producing ActRIIB fusion polypeptides, see Gene Expression Systems, Academic Press (Fernandez et al. eds. 1999). Any cell line compatible with the present invention may be used to produce the presently disclosed ActRIIB fusion polypeptides.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids or viral, e.g., phage, or phagemid, as appropriate. For further details see, for example, Sambrook et al. (1989) Molecular Cloning: A Laboratory Manual, 2nd ed., Cold Spring Harbor Laboratory Press. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, 2nd ed., John Wiley & Sons (Ausubel et al eds. 1992).

Thus, a further aspect of the present invention is a host cell containing nucleic acid as disclosed herein. Additionally, the invention provides a method comprising introducing such nucleic acid into a host cell. The introduction may employ any suitable technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage.

The introduction may be followed by causing or allowing expression from the nucleic acid, e.g., by culturing host cells under conditions appropriate for expression of the nucleic acid.

### IV. Methods for Identifying Inhibitors

Yet another aspect of the invention provides a method of identifying therapeutic agents useful in treatment of muscle and bone disorders. Appropriate screening assays, e.g., ELISA-based assays, are known in the art. In such a screening assay, a first binding mixture is formed by combining an ActRIIB fusion polypeptide and a ligand, e.g., GDF-8, BMP-11, activin; and the amount of binding in the first binding mixture (M₀) is measured. A second binding mixture is also formed by combining an ActRIIB fusion polypeptide, the ligand, and the compound or agent to be screened, and the amount of binding in the second binding mixture (M₁) is measured. The amounts of binding in the first and second binding mixtures are then compared, for example, by calculating the M₁/M₀ ratio. The compound or agent is considered to be capable of inhibiting ActRIIB-mediated cell signaling if a decrease in binding in the second binding mixture as compared to the first binding mixture is observed. The formulation and optimization of binding mixtures is within the level of skill in the art, such binding mixtures may also contain buffers and salts necessary to enhance or to optimize binding, and additional control assays may be included in the screening assay of the invention.

Compounds found to reduce the ActRIIB fusion polypeptide-ligand binding by at least about 10% (i.e., M₁/M₀<0.9), preferably greater than about 30%, may thus be identified and then, if desired, secondarily screened for the capacity to inhibit GDF-8 activity in other assays, such as the ActRIIB binding assay, and other cell-based and in vivo assays as described in Examples 5-12.

V. Methods of Treating Disease and Other Uses

The presently disclosed ActRIIB fusion polypeptides are soluble and possess pharmacokinetic properties that make them suitable as therapeutic agents, i.e., useful to prevent, diagnose, or treat various medical disorders in animals, and especially, humans. In certain embodiments, circulatory half-life of the ActRIIB fusion polypeptide exceeds 5, 7, 10, or 14 days.

The ActRIIB fusion polypeptides can be used to inhibit one or more activities of GDF-8 associated with muscle and/or bone disorders. Inhibition of GDF-8 activity can be measured in pGL3(CAGA)₁₂ reporter gene assays (RGA) as described in Thies et al. (Growth Factors (2001) 18:251-259) or as illustrated in Example 6.

The medical disorder being diagnosed, treated, or prevented by the presently disclosed ActRIIB fusion polypeptides is a muscle or neuromuscular disorder; an adipose tissue disorder such as obesity; type 2 diabetes; impaired glucose tolerance; metabolic syndromes (e.g., syndrome X); insulin resistance induced by trauma such as burns or nitrogen imbalance; or bone degenerative disease such as osteoporosis.

The presently disclosed ActRIIB fusion polypeptides may also be used in therapies to repair damaged muscle, e.g., myocardium, diaphragm, etc. Exemplary disease and disorders further include muscle and neuromuscular disorders such as muscular dystrophy (including Duchenne's muscular dystrophy); amyotrophic lateral sclerosis (ALS), muscle atrophy; organ atrophy; frailty; carpal tunnel syndrome; congestive obstructive pulmonary disease; and sarcopenia, cachexia and other muscle wasting syndromes.

Other medical disorders being diagnosed, treated, or prevented by the presently disclosed ActRIIB fusion polypeptides are disorders associated with a loss of bone, which include osteoporosis, especially in the elderly and/or postmenopausal women; glucocorticoid-induced osteoporosis; osteopenia; osteoarthritis; and osteoporosis-related fractures. Other target metabolic bone diseases and disorders include low bone mass due to chronic glucocorticoid therapy, premature gonadal failure, androgen suppression, vitamin D deficiency, secondary hyperparathyroidism, nutritional deficiencies, and anorexia nervosa. The ActRIIB fusion polypeptides are preferably used to prevent, diagnose, or treat such medical disorders in mammals, especially, in humans.

Compositions comprising the ActRIIB fusion polypeptides of the present invention are administered in therapeutically effective amounts. Generally, a therapeutically effective amount may vary with the subjects age, condition, and sex, as well as the severity of the medical condition in the subject. The dosage may be determined by a physician and adjusted, as necessary, to suit observed effects of the treatment. Generally, the compositions are administered so that polypeptides are given at a dose from 1 μg/kg to 20 mg/kg, 1 μg/kg to 10 mg/kg, 1 μg/kg to 1 mg/kg, 10 μg/kg to 1 mg/kg, 10 μg/kg to 100 μg/kg, 100 μg to 1 mg/kg, and 50 μg/kg to 1 mg/kg, or as described in Examples 10 and 11. The compositions may be given as a bolus dose, to maximize the circulating levels for the greatest length of time after the dose. Continuous infusion may also be used after the bolus dose.

The specification for the dosage unit polypeptides of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription-based assays, GDF-8 binding assays, creatine kinase assays, assays based on the differentiation of pre-adipocytes, assays based on glucose uptake in adipocytes, and immunological assays.

As a further aspect of the invention, the ActRIIB fusion polypeptides of the present invention may be used to detect the presence of proteins belonging to the TGF-β superfamily, such as BMP-11 and GDF-8, in vivo or in vitro. By correlating the presence or level of these proteins with a medical condition, one of skill in the art can diagnose the associated medical condition. The medical conditions that may be diagnosed by the presently disclosed ActRIIB fusion polypeptides are set forth above.

Such detection methods are well known in the art and include ELISA, radioimmunoassay, immunoblot, Western blot, immunofluorescence, immunoprecipitation, and other comparable techniques. The polypeptides may further be provided in a diagnostic kit that incorporates one or more of these techniques to detect a protein (e.g., GDF-8). Such a kit may contain other components, packaging, instructions, or other material to aid the detection of the protein and use of the kit.

Where the ActRIIB fusion polypeptides are intended for diagnostic purposes, it may be desirable to modify them, for example, with a ligand group (such as biotin) or a detectable marker group (such as a fluorescent group, a radioisotope or an enzyme). If desired, the ActRIIB fusion polypeptides may be labeled using conventional techniques. Suitable labels include fluorophores, chromophores, radioactive atoms, electron-dense reagents, enzymes, and ligands having specific binding partners. Enzymes are typically detected by their activity. For example, horseradish peroxidase can be detected by its ability to convert tetramethylbenzidine (TMB) to a blue pigment, quantifiable with a spectrophotometer. Other suitable binding partners include biotin and avidin or streptavidin, IgG and protein A, and the numerous receptor-ligand couples known in the art. Other permutations and possibilities will be readily apparent to those of ordinary skill in the art, and are considered as equivalents within the scope of the instant invention.

### VI. Pharmaceutical Compositions and Methods of Administration

The present invention provides compositions suitable for administration to patients. The compositions typically comprise one or more ActRIIB fusion polypeptides of the invention and a pharmaceutically acceptable excipient. As used herein, the phrase “pharmaceutically acceptable excipient” refers to any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. The administration may, for example, be intravenous, intraperitoneal, intramuscular, intracavity, subcutaneous or transdermal. It may also be possible to obtain compositions that may be topically or orally administered.

Solutions or suspensions used for intradermal or subcutaneous application typically include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor™ EL (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible carrier. They can be enclosed in gelatin capsules or compressed into tablets. For the purpose of oral therapeutic administration, the ActRIIB fusion polypeptides can be incorporated with excipients and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

For administration by inhalation, the ActRIIB fusion polypeptides are delivered in: the form of an aerosol spray from pressured container or dispenser which contains a suitable propellant, e.g., a gas such as carbon dioxide, or a nebulizer.

Systemic administration can also be by transmucosal or transdermal means. For example, in the case of ActRIIB-Fc, compositions may be capable of transmission across mucous membranes (e.g., intestine, mouth, or lungs) via the FcRn receptor-mediated pathway (U.S. Pat. No. 6,030,613). Transmucosal administration can be accomplished, for example, through the use of lozenges, nasal sprays, inhalers, or suppositories. For transdermal administration, the active compounds are formulated into ointments, salves, gels, or creams as generally known in the art. For transmucosal or transdermal administration, penetrants appropriate to the barrier to be permeated are used in the formulation. Such penetrants are generally known in the art, and include, for example, detergents, bile salts, and fusidic acid derivatives.

The presently disclosed ActRIIB fusion polypeptides can prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions containing the presently disclosed ActRIIB fusion polypeptides can also be used as pharmaceutically acceptable carriers. These can be prepared according to methods known to those skilled in the art, for example, as described in U.S. Pat. No. 4,522,811.

It is may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Nucleic acids encoding ActRIIB fusion polypeptides, such as the nucleic acids described above, can be introduced to a cell within tissue, an organ, or an organism so that the encoded polypeptides can then be expressed. This methodology may be useful, for example, in evaluating effects of ActRIIB fusion polypeptides on individual tissues and organs. In certain embodiments, nucleic acid encoding an ActRIIB fusion polypeptide is linked to a tissue-specific expression control sequence, e.g., muscle-specific promoter sequence such as the myosin promoter or the desmin promoter, the muscle-specific enhancer elements such as the muscle creatine kinase enhancer. Those of skill in the art will recognize that specific polynucleotide sequences can be inserted into the viral or plasmid vectors that can be injected into a mammal systemically, or locally. Host cells may also be harvested, and a nucleic acid encoding an ActRIIB fusion polypeptide may be transfected into such cells ex vivo for subsequent reimplantation using methods known in the art. Nucleic acids may be also transfected into a single cell embryo to create a transgenic animal as described in Gene Expression Systems, Academic Press (Fernandez et al. eds. 1999).

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with a true scope and spirit of the invention being indicated by the appended claims.

The following examples illustrate some embodiments and aspects of the invention. One of ordinary skill in the art will recognize the numerous modifications and variations that may be performed without altering the spirit or scope of the present invention. Such modifications and variations are encompassed within the scope of the invention. The examples do not in any way limit the invention.

### EXAMPLES

### Example 1

### Purification of GDF-8

Conditioned media from a selected cell line expressing recombinant human GDF-8 protein (mature GDF-8 and GDF-8 propeptide) was acidified to pH 6.5 and applied to a 80×50 mm POROS™ HW anion exchange column in tandem to a 80×50 mm POROS™ SP cation exchange column (PerSeptive Biosystems, Foster City, Calif.). The flow through was adjusted to pH 5.0 and applied to a 75×20 mm POROS™ SP cation exchange column (PerSeptive Biosystems) and eluted with a TFA/acetonitrile gradient. Fractions containing the GDF-8 latent complex, as confirmed by SDS-PAGE, were pooled, acidified with trifluoroacetic acid (TFA) to pH 2-3, then brought up to 200 ml with 0.1% TFA to lower the viscosity. The pool was then applied to a 250×21.2 mm C₅ column (Phenomenex, Torrance, Calif.) preceded by a 60×21.2 mm guard column (Phenomenex) and eluted with a TFA/acetonitrile gradient, to separate mature GDF-8 from GDF-8 propeptide. Pooled fractions containing mature GDF-8 were concentrated by lyophilization to remove the acetonitrile and 20 ml of 0.1% TFA was added. The sample was then applied to a 250×10 mm C₅ column (Phenomenex) heated to 60° C. to aid in separation. This was repeated until further separation could no longer be achieved. Fractions containing mature GDF-8 were then pooled and brought up to 40% acetonitrile and applied to a 600×21.2 BioSep™ S-3000 size exclusion column (Phenomenex) preceded by a 60×21.2 guard column. Fractions containing purified mature GDF-8 and the GDF-8 propeptide were separately pooled and concentrated for use in subsequent experiments.

On SDS-PAGE, purified mature GDF-8 migrated as a broad band at 25 kDa under non-reducing conditions and 13 kDa under reducing conditions. Under reducing and non-reducing conditions, BMP-11 propeptide migrated at around 35 kDa. A similar SDS-PAGE profile has been reported for murine GDF-8 by McPherron et al. (Proc. Natl. Acad. Sci. U.S.A. (1997) 94:12457-12461) and reflects the dimeric nature of the mature protein. The GDF-8 propeptide migrated at about 35 kDa on reducing SDS-PAGE. Active mature BMP-11 dimer was purified from conditioned media from a cell line expressing recombinant human BMP-11 in the same manner. The conditioned media was loaded onto a 10 ml TALON™ column (Clonetech, Palo Alto, Calif.). The bound protein was eluted with 50 mM Tris pH 8.0/1 M NaCl/500 mM imidazole. Fractions containing the BMP-11 complex were pooled and acidified with 10% trifluoroacetic acid to a pH of 3. The BMP-11 complex pool was applied to a −250×4.6 mm Jupiter C4 column (Phenomenex), which was heated to 60° C. for better separation of the mature BMP-11 and BMP-11 propeptide. BMP-11 was eluted with a TFA/acetonitrile gradient. The fractions containing BMP-11 were concentrated by lyophilization to remove the acetonitrile.

### Example 2

### Characteristics of Purified Recombinant Human GDF-8

50 μg of each purified mature GDF-8 and purified GDF-8 propeptide were mixed and dialyzed into 50 mM sodium phosphate, pH 7.0, and chromatographed on a 300×7.8 mm BioSep™ S-3000 size exclusion column (Phenomenex). Molecular weight of the mature GDF-8/propeptide complex was determined from elution time, using molecular weight standards (Bio-Rad Laboratories, Hercules, Calif.) chromatographed on the same column.

When purified GDF-8 propeptide was incubated with purified mature GDF-8 at neutral pH, the two proteins appeared to complex, as indicated by the size exclusion profile. The primary protein peak eluted at 12.7 minutes had an estimated molecular weight of 78 kDa from molecular weight standards (Bio-Rad Laboratories) chromatographed on the same column. The size of the complex was most consistent with one dimer of the mature GDF-8 associating with two monomers of propeptide.

To confirm this observation, a preparation containing both mature GDF-8 and GDF-8 propeptide was incubated with or without 100 mM 1-Ethyl 3-(3-dimethylaminopropyl)carbodiamide hydrochloride (EDC, Pierce Chemical, Rockford, Ill.) for 1 hour at room temperature, acidified with HCl to pH 2-3, and concentrated with a Micron-10 Amicon concentrator (Millipore, Bedford, Mass.) for SDS-PAGE, using a tricine buffered 10% acrylamide gel. Proteins were visualized by Coomassie™ blue staining of SDS-PAGE.

### Example 3

### Expression of ActRIIB-Fc in CHO Cells

A full-length human ActRIIB cDNA was used to PCR-clone the extracellular domain (excluding the sequence encoding the signal peptide). The primers used were flanked by SpeI (5′) and NotI (3′) sites. Following PCR amplification, this PCR fragment was cloned into the SpeI/NotI sites of the expression plasmid pHTop-HBML/EKFc. The open reading frame encodes: honeybee mellitin leader (amino acids 1 to 21 of SEQ ID NO:3); human ActRIIB extracellular domain (amino acids 23 to 138 of SEQ ID NO:3); enterokinase cleavage site (DDDK, SEQ ID NO:6); and human IgG₁ Fc fragment (amino acids 148 to 378 of SEQ ID NO:3). As a result of the insertion of the SpeI site, there was an addition of Thr-22 in the sequence.

A CHO stable cell line stably transfected to express the above ActRIIB-Fc was obtained by lipofectin transfection of the pHTop-HBML vector containing the ActRIIB-Fc construct into CHO/A2 cells. Transfected cells were selected in 0.1 μM methotrexate. Western blot analysis of conditioned media was used to identify the highest expressing clones.

The pHTop vector was derived from pED (Kaufman et al. (1991) Nucleic Acids Res. 19:4485-4490) by removing the majority of the adeno major late promoter and inserting six repeats of the tet operator as described in Gossen et al. (1992) Proc. Natl. Acad. Sci. U.S.A. 89:5547-5551.

The CHO/A2 cell line was derived from CHO DUKX B11 cells (Urlaub et al. (1980) Proc. Natl. Acad. Sci. U.S.A. 77:4216-4220) by stably integrating a transcriptional activator, a fusion protein between the tet repressor fused to the herpes virus VP16 transcriptional domain (Gossen et al. (1992) Proc. Natl. Acad. Sci. U.S.A. 89:5547-5551).

### Example 4

### Purification of ActRIIB-Fc

Raw material concentrated from conditioned medium was purified by rProtein A Sephadex Fast Flow™ (XK26/4.5 cm, 23.8 ml; Pharmacia, Piscataway, N.J.) to 99% purity as determined by size exclusion chromatography as follows. Frozen conditioned medium was thawed at 37° C. water bath and filtered through 0.22 μm filters. Four parts of the filtered solution was mixed with one part of Protein A loading buffer (0.65 M Na₂SO₄, 20 mM sodium citrate, 20 mM boric acid, 20 mM Na₂HPO₄, pH 9.0) and ran over the protein A column at room temperature. ActRIIB-Fc was eluted off the column using Protein A eluting buffer (0.15 M NaCl, 20 mM citric acid, pH 2.5) with gradient or step up at pH around 4-5, and the peak was collected and neutralized to pH 7.0 by adding 25% neutralization buffer (0.05 M Na₂HPO₄, 0.15 M NaCl, pH 7.2). The fractions were evaluated by size exclusion chromatography and SDS-PAGE, and then pooled and stored at 4° C. The purified protein was formulated into PBS by Sephadex™ G-25 desalting column (XK50/13.4 cm, 236 ml, Pharmacia), and then filtered through a 0.22 μm filer and stored at 4° C.

### Example 5

### Binding Properties of Purified GDF-8 and BMP-11 in ActRIIB-Fc Binding Assay

The GDF-8 latent complex was biotinylated at a ratio of 20 moles of EZ-link™ Sulfo-NHS-Biotin (Pierce Chemical, Cat. No. 21217) to 1 mole of the GDF-8 complex for 2 hours on ice, inactivated with 0.5% TFA, and subjected to chromatography on a C4 Jupiter 250×4.6 mm column (Phenomenex) to separate mature GDF-8 from GDF-8 propeptide. Biotinylated mature GDF-8 fractions eluted with a TFA/acetonitrile gradient were pooled, concentrated and quantified by MicroBCA™ protein Assay Reagent Kit (Pierce Chemical, Cat. No. 23235).

Biotinylated mature BMP-11 was prepared from BMP-11 latent complex in the same manner as described above. Recombinant ActRIIB-Fc, prepared as described in Examples 3 and 4, was coated on 96-well flat-bottom assay plates (Costar, N.Y., Cat. No. 3590) at 1 μg/ml in 0.2 M sodium carbonate buffer (pH 10) overnight at 4° C. Plates were then blocked with 1 mg/ml bovine serum albumin and washed following standard ELISA protocol. 100 μl aliquots of biotinylated GDF-8 or BMP-11 at various concentrations were added to the blocked ELISA plate, incubated for 1 hr and washed. The amount of bound GDF-8 or BMP-11 was detected by Streptavidin-Horseradish peroxidase (SA-HRP, BD PharMingen, San Diego, Calif., Cat. No. 13047E) followed by the addition of TMB (KPL, Gaithersburg, Md., Cat. No. 50-76-04). Colorimetric measurements were done at 450 nM in a Molecular Devices microplate reader.

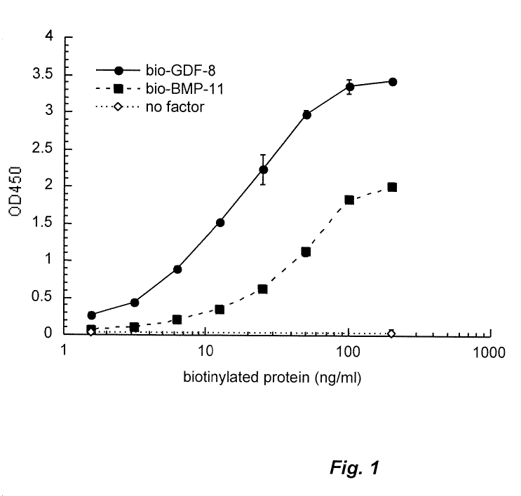

As shown in FIG. 1, biotinylated GDF-8 and BMP-11 bound to ActRIIB-Fc, with an ED₅₀ of 15 ng/ml and 40 ng/ml, respectively.

### Example 6

### Inhibitory Activity of ActRIIB-Fc in Reporter Gene Assay

To demonstrate the activity of ActRIIB-Fc, a reporter gene assay (RGA) was developed using a reporter vector PGL3(CAGA)₁₂ sequence coupled luciferase. The CA5A sequence was previously reported to be a TGF-β responsive sequence within the promoter of the TGF-β induced gene PAI-1 (Denner et al. (1998) EMBO J. 17:3091-3100).

A reporter vector containing 12 CAGA boxes was made using the basic reporter plasmid PGL3 (Promega, Madison, Wis.). The TATA box and transcription initiation site from the adenovirus major later promoter (−35/+10) was inserted between the BgIII and HindIII sites. Oligonucleotides containing 12 repeats of the CAGA boxes, AGCCAGACA, were annealed and cloned into the XhoI site. The human rhabdomyosarcoma cell line A204 (ATCC HTB-82) was transiently transfected with pGL3(CAGA)₁₂ using FuGENE™ 6 transfection reagent (Boehringer Manheim, Germany). Following transfection, cells were cultured on 48 well plates in McCoy's 5A medium supplemented with 2 mM glutamine, 100 U/ml streptomycin, 1000 μg/ml penicillin and 10% fetal calf serum for 16 hrs. Cells were then treated with or without 10 ng/ml GDF-8 and various concentration of ActRIIB-Fc in McCoy's 5A media with glutamine, streptomycin, penicillin, and 1 mg/ml bovine serum albumin for 6 hrs at 37° C. Luciferase was quantified in the treated cells using the Luciferase Assay System (Promega).

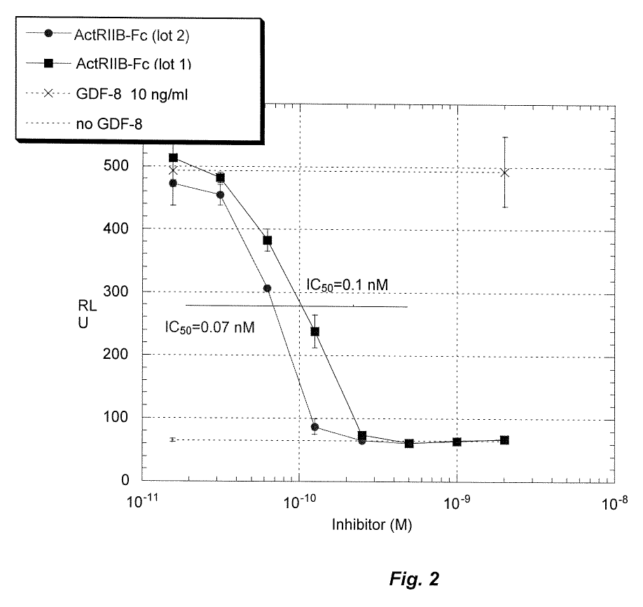

Two independently purified lots of ActRIIB showed an IC₅₀ from 0.07 to 0.1 nM in the above reporter gene assay (FIG. 2).

### Example 7

### Pharmacokinetics

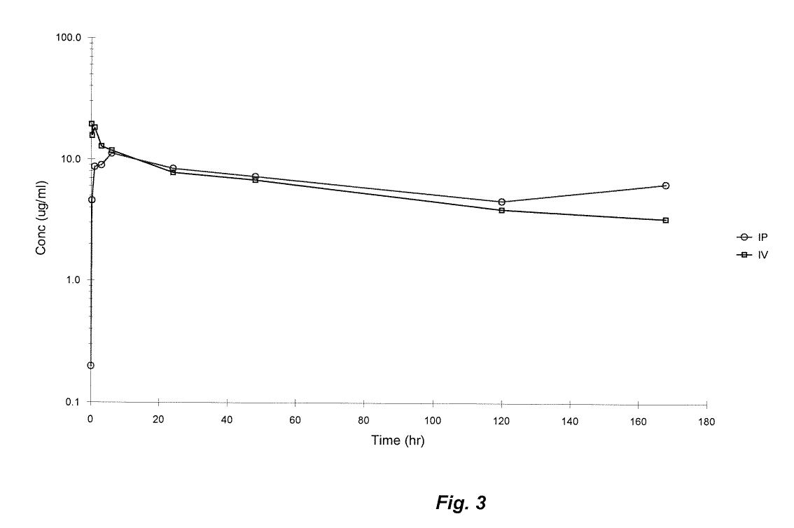

The pharmacokinetics (PK) of ActRIIB-Fc was evaluated in C57B6/SCID mice (The Jackson Laboratory, Bar Harbor, Me.) at a dose of 1 mg/kg as a single intravenous (IV) or intraperitoneal (IP) administration. ActRIIB-Fc, produced and purified as described in Examples 3 and 4, was radiolabeled using the iodogen method (Protein Pharmacokinetics and Metabolism, Plenum Press, New York, N.Y. (Ferraiolo et al. eds. 1992)). The animals received a mixture of unlabeled and ¹²⁵I labeled ActRIIB-Fc at the dose listed above and serum concentrations were determined based on ¹²⁵I radioactivity in the serum and the specific activity of the injected dose. FIG. 3 shows the serum concentration based on TCA-precipitated counts versus time for ActRIIB-Fc administered either IV or IP. Absorption from IP injection was complete, and bioavailability was close to 100% within the first 180 hr post injection; the initial volume distribution matched mouse plasma volume (50 ml/kg); peak serum concentration was 11 μg/ml (IP, at 6 hr post injection) and 19.4 μg/ml (IV); half-life during the terminal elimination phase was about 5 days.

### Example 8

### In vivo Effect of ActRIIB-Fc

In order to determine if ActRIIB increases muscle mass in adult mice, an in vivo study on was conducted with seven-week-old female C57B6/SCID (The Jackson Laboratory). Mice were weighed and evenly distributed with respect to body weight into groups of eight. During a four-week study, each group received a weekly intraperitoneal injection of the following: ActRIIB-Fc (60 mg/kg, 3 mg/kg, or 60 μg/kg), mouse monoclonal anti-GDF-8 antibody JA16 (60 mg/kg), or PBS buffer (vehicle control). JA16 was chosen because this antibody is specific for GDF-8, and was shown to inhibit the muscle-down regulatory activity of GDF-8 in vivo, in a separate study (U.S. Patent App. Pub. No. 20030138422). Animals were assessed for gain in lean body mass by subjecting them to dexascan analysis before and after the treatment period. Muscle mass was assessed by dissecting and weighing the gastrocnemius and quadriceps. The peri-uterine fat pad was also removed and weighed. Spleen and thymus weights were also measured.

The results of this study indicated that ActRIIB-Fc significantly inhibited GDF-8 activity in vivo resulting in increased muscle mass. As anticipated, mice administered JA16 exhibited slightly higher body and skeletal muscle weights and had a statistically significant (p=0.05) increase in quadriceps weights (Table 4). The treatments with 60 and 3 mg/kg ActRIIB-Fc were surprisingly significantly more effective as compared to the JA16 antibody. The groups administered 60 mg/kg ActRIIB-Fc and 3 mg/kg ActRIIB-Fc had about 3 and 2 times increased body weights respectively as compared to the controls (Table 1). These increases were first observed after one dose. The quadriceps muscle weights, as absolute weights, were increased in the mice administered 60 and 3 mg/kg ActRIIB-Fc (Table 3). The gastrocnemius muscles, as absolute weights, were increased in mice administered 60 mg/kg JA16 and 60 or 3 mg/kg ActRIIB-Fc (Table 3). As a percent of body weight, quadriceps muscle weights were increased in the same three treatment groups compared to controls (Table 4). Also, as a percent of body weight, the gastrocnemius weight was increased in the mice treated with 60 mg/kg ActRIIB-Fc (Table 4).

TABLE 1
Terminal Body Weights

| | | JA16 | ActRIIB | | ActRIIB |
|---|---|---|---|---|---|
| | Control | 60 mg/kg | 60 mg/kg | ActRIIB 3 mg/kg | 60 μg/kg |
| Body | 20.2 ± 1.76 | 20.9 ± 1.12 | 25.0 ± 1.90* | 22.5 ± 2.35* | 20.8 ± 1.97 |
| Weight | | | | | |
| (g) ± SD | | | | | |

*= Group Difference at p = 0.05 compared to controls

TABLE 2
Absolute Weight Gain

| | | JA16 | ActRIIB | ActRIIB | ActRIIB |
|---|---|---|---|---|---|
| | Control | 60 mg/kg | 60 mg/kg | 3 mg/kg | 60 μg/kg |
| Body | 1.99 ± 1.123 | 2.62 ± 1.007 | 6.23 ± 1.126* | 4.28 ± 1.748* | 1.24 ± 1.010 |
| Weight | | | | | |
| (g) ± SD | | | | | |

*= Group Difference at p = 0.05 compared to controls

TABLE 3
Absolute Organ Weights (g)

| | | JA16 | ActRIIB | ActRIIB | ActRIIB |
|---|---|---|---|---|---|
| | Control | 60 mg/kg | 60 mg/kg | 3 mg/kg | 60 μg/kg |
| Spleen | 0.044 | 0.025* | 0.060 | 0.071 | 0.059 |
| Thymus | 0.0342 | 0.178* | 0.0260 | 0.0333 | 0.0344 |
| Quadriceps | 0.151 | 0.171 | 0.232** | 0.193** | 0.159 |
| Gastroc. | 0.111 | 0.123* | 0.156** | 0.133* | 0.112 |

*= Group Difference at p = 0.05 compared to controls
**= Group Difference at p = 0.01 compared to controls

TABLE 4
Organ Weights as Percent of Body Weight

| | | JA16 | ActRIIB | ActRIIB | ActRIIB |
|---|---|---|---|---|---|
| | Control | 60 mg/kg | 60 mg/kg | 3 mg/kg | 60 μg/kg |
| Spleen | 0.214 | 0.119* | 0.227 | 0.298 | 0.273 |
| Thymus | 0.1628 | 0.0850* | 0.0992 | 0.1391 | 0.1569 |
| Quadriceps | 0.749 | 0.820* | 0.926** | 0.861** | 0.768 |
| Gastrocnemius | 0.548 | 0.590 | 0.621** | 0.593 | 0.540 |

*= Group Difference at p = 0.05 compared to controls
**= Group Difference at p = 0.01 compared to controls

### Example 9

### Dose-Dependent Effect of ActRIIB-Fc on Muscle Mass

To further investigate the effect of ActRIIB-Fc on muscle mass in adult mice, a study on was conducted with seven-week-old female C57B6/SCID (The Jackson Laboratory). Mice were weighed and evenly distributed with respect to body weight into four groups of six (6 SCID, 6 C57 mice, and two control groups of 6 mice each). Each group received a weekly intraperitoneal injection of 60 mg/kg ActRIIB-Fc or PBS buffer (vehicle control) for one to four weeks. On day 29 of the study, animals were assessed for muscle mass was assessed by dissecting and weighing the gastrocnemius and quadriceps. The results of this study indicated that ActRIIB-Fc significantly inhibited GDF-8 activity in vivo resulting in increased muscle mass even after a single administration of ActRIIB as compared to the vehicle control. The quadriceps muscle weights, as absolute weights, were increased in both C57 and SCID mice by 21% to 60% (Table 5). Likewise, the gastrocnemius muscle mass, as absolute weights, was increased by 31 to 51% (Table 5).

TABLE 5
Increase in Muscle Mass Following One or More Doses of
ActRIIB-Fc

| | ActRIIB | ActRIIB | ActRIIB |
|---|---|---|---|
| | 1 dose | 2 doses | 4 doses |
| Quadriceps (SCID) | 21% | 60% | 44% |
| Gastrocnemius (SCID) | 47% | 36% | 31% |
| Quadriceps (C57) | 41% | | 65% |
| Gastrocnemius (C57) | 37% | | 51% |

### Example 10

### In vivo Role of GDF-8 in Trabecular Bone

Inhibition of GDF-8 increases muscle mass. Increased mechanical loading, either due to increased muscle activity or increased body weight, is associated with increased bone mass and bone density. Therefore, GDF-8 knockout (KO) mice were assessed for altered bone mass and microarchitecture. An initial assessment of adult mice showed that bone density in the spine of the KO mice was nearly two-fold higher than that of their wild-type littermates. This increase far exceeded what might have been expected to be solely due to the increased muscle mass in the GDF-8 KO mice.

High resolution microtomographic imaging (μCT40, Scanco Medical, Switzerland) was used to assess the trabecular bone volume fraction and microarchitecture in the 5th lumbar vertebrae and distal femora and cortical bone geometry at the femoral mid-diaphysis of adult GDF-8 wildtype (WT) and KO mice. Specimens were taken from 9-10 month old GDF-8 KO and littermate controls (four mice of each genotype and sex). The entire vertebral body and femur were scanned using microcomputed tomography at 12 μm resolution. Regions of interest encompassing the trabecular bone of the vertebral body or the trabecular bone of the distal femoral metaphysis (i.e., secondary spongiosa) were identified using a semi-automated contouring algorithm. The following parameters were computed using direct 3D assessments: bone volume fraction (%), trabecular thickness (μm), separation (μm) and number (1/mm). In addition, the connectivity density, an indicator of how well the trabecular network is connected, was assessed as well as cortical bone parameters at the middiaphyseal region in the femur, including total area, bone area, and cortical thickness.

Both male and female KO mice had dramatically increased trabecular bone density in the vertebral body compared to WT littermates (n=4, +93% and +70%, respectively, p<0.0001). This increased trabecular bone density was accompanied by a 14% increase in trabecular thickness (p=0.03), a 38% increase in trabecular number (p=0.0002), and a 10% decrease in trabecular separation (p=0.009). The combined effect of these changes in architecture and density resulted in a 3.4- and 1.7-fold increase in connectivity in male and female KO, respectively, compared to their WT littermates (p<0.0001). In addition, a rough measure of the level of mineralization of the trabecular bone indicated that the average mineral content of the trabeculae was 8% higher in the KO mice relative to the controls (p<0.0001). There is a hint that the effect is larger in male than female mice, but the sample size is too small to make definitive conclusions. Vertebral trabecular bone characteristics assessed by high-resolution microcomputed tomography are shown in Table 6.

In contrast to observations in the spine, male and female KO mice had lower trabecular bone density in the distal femur than WT littermates (n=4, p=0.05 for overall genotype effect) (Table 7). This decrement in bone density was more pronounced in female KO than in male KO mice. GDF-8 KO mice had similar trabecular thickness as their WT littermates, but had fewer trabeculae and increased trabecular separation compared to littermate controls. However, although cortical thickness at the femoral midshaft was similar in male GDF-8 KO and their littermate controls, it was approximately 10% greater in the GDF-8 KO female mice than their WT littermates (n=4, p=0.04) (see Table 8). There were no differences in cortical bone area or bone area fraction between the two genotypes.

TABLE 6
Vertebral Trabecular Bone Characteristics (Mean ± SEM)

| | Male WT | Male KO | Female WT | Female KO |
|---|---|---|---|---|
| Bone volume fraction (%) | 23.3 ± 4.7 | 45.0 ± 5.5 | 27.5 ± 5.5 | 46.9 ± 10.8 |
| Trabecular thickness (μm) | 52 ± 3 | 58 ± 6 | 52 ± 5 | 61 ± 8 |
| Trabecular separation (μm) | 210 ± 21 | 145 ± 8 | 183 ± 21 | 169 ± 41 |
| Trabecular number (1/mm) | 4.6 ± 0.4 | 7.0 ± 0.4 | 5.2 ± 0.4 | 6.6 ± 1.3 |
| Connectivity density (1/mm³) | 137 ± 15 | 470 ± 114 | 198 ± 29 | 339 ± 81 |
| Degree of anisotropy | 1.68 ± 0.08 | 1.29 ± 0.02 | 1.54 ± 0.12 | 1.34 ± 0.03 |

TABLE 7
Characteristics of the Trabecular Bone in Distal Femoral
Metaphysis (Mean ± SEM)

| | Male WT | Male KO | Female WT | Female KO |
|---|---|---|---|---|
| Bone volume fraction (%) | 5.1 ± 1.8 | 2.9 ± 1.7 | 11.9 ± 7.0 | 5.4 ± 3.1 |
| Trabecular thickness (μm) | 68 ± 1.2 | 68 ± 2.7 | 73 ± 7 | 63 ± 9 |
| Trabecular separation (μm) | 353 ± 16 | 472 ± 90 | 296 ± 73 | 464 ± 98 |
| Trabecular number (1/mm) | 2.84 ± 0.12 | 2.24 ± 0.51 | 3.46 ± 0.69 | 2.26 ± 0.57 |
| Connectivity density (1/mm³) | 5.9 ± 3.7 | 4.0 ± 6.9 | 31.5 ± 25.2 | 15.4 ± 15.1 |

TABLE 8
Characteristics of the Cortical Bone at the Femoral Mid-Diaphysis
(Mean ± SEM)

| | Male WT | Male KO | Female WT | Female KO |
|---|---|---|---|---|
| Bone Area (mm²) | 5.1 ± 1.8 | 2.9 ± 1.7 | 11.9 ± 7.0 | 5.4 ± 3.1 |
| Cortical Thickness | 68 ± 1.2 | 68 ± 2.7 | 73 ± 7 | 63 ± 9 |
| (μm) | | | | |
| Bone Area/Total | 353 ± 16 | 472 ± 90 | 296 ± 73 | 464 ± 98 |
| Area (%) | | | | |

### Example 11

### Treatment of Muscle and Bone Degenerative Disorders

Inhibitors of GDF-8 such as, for example, ActRIIB fusion polypeptides are useful for treatments directed at increased muscle mass, and also for prevention and treatment of osteoporosis. In addition, inhibition of GDF-8 may be useful in other instances where a bone anabolic effect is desired, such as augmentation of bone healing (i.e., fracture repair, spine fusion, etc.). The ActRIIB fusion polypeptides of the invention are used to treat a subject at disease onset or having an established muscle or bone degenerative disease.

Efficacy of ActRIIB-Fc for treatment of bone disorders, e.g., osteoporosis, is confirmed using well-established models of osteoporosis. For example, ovariectomized mice have been used to test the efficacy of new osteoporosis drug treatments (Alexander et al. (2001) J. Bone Min. Res. 16:1665-1673; and Anderson et al. (2001) J. Endocrinol. 170:529-537). Similar to humans, these rodents exhibit a rapid loss of bone following ovariectomy, especially in cancellous bone. Outcome assessments are based on bone mineral density, biochemical markers of bone turnover in serum and urine, bone strength, and histology/histomorphometry.

In one study, normal and/or immune compromised female mice are ovariectomized at 12-16 weeks of age and allowed to lose bone for four to six weeks. Following this bone loss period, treatment with ActRIIB-Fc (IP injection) or vehicle is conducted for one to six months. The treatment protocol could vary, with testing of different doses and treatment regimens (e.g., daily, weekly, or bi-weekly injections). It is anticipated that untreated ovariectomized mice (or rats) would lose approximately 10-30% of bone density relative to intact (i.e., non-ovariectomized), age-matched mice. It is anticipated that mice treated with ActRIIB-Fc would have 10 to 50% greater bone mass and bone density than those mice receiving vehicle treatment, and moreover that this increase in bone density would be associated with increased bone strength, particularly in regions with a greater proportion of cancellous bone compared to cortical bone.

The goal of another study is to demonstrate that ActRIIB-Fc is effective in preventing the decline in bone mass, microarchitecture and strength associated with estrogen deficiency. Thus, the study has a similar design to the one described above, except that treatment with ActRIIB-Fc antibody would be initiated immediately after ovariectomy, rather than after the bone loss period. It is anticipated that mice treated with ActIIB-Fc would lose significantly less bone mass following ovariectomy than mice treated with vehicle.

The ActRIIB fusion polypeptides are also used to prevent and/or to reduce severity and/or the symptoms of the disease. It is anticipated that the ActRIIB fusion polypeptides would be administered as a subcutaneous injection as frequently as once per day and as infrequently as once per month. Treatment duration could range from one month and several years.

To test the clinical efficacy of ActRIIB-Fc in humans, postmenopausal women with low bone mass are identified by bone density testing and randomized to a treatment group. Treatment groups include a placebo group and one to three groups receiving antibody (different doses). Individuals are followed prospectively for one to three years to assess changes in biochemical markers of bone turnover, changes in bone mineral density, and the occurrence of fragility fractures. It is anticipated that individuals receiving treatment would exhibit an increase in bone mineral density in the proximal femur and lumbar spine of 2-30% relative to baseline, and would have a decreased incidence of fragility fractures. It is anticipated that biochemical markers of bone formation would increase.

The polypeptides are administered as the sole active compound or in combination with another compound or composition. When administered as the sole active compound or in combination with another compound or composition, the dosage is preferably from approximately 1 μg/kg and 20 mg/kg, depending on the severity of the symptoms and the progression of the disease. The appropriate effective dose is selected by a treating clinician from the following ranges: 1 μg/kg to 20 mg/kg, 1 μg/kg to 10 mg/kg, 1 μg/kg to 1 mg/kg, 10 μg/kg to 1 mg/kg, 10 μg/kg to 100 μg/kg, 100 μg to 1 mg/kg, and 500 μg/kg to 1 mg/kg. Exemplary treatment regimens and outcomes are summarized in Table 9. Alternative regimens include: (1) 1×IC₅₀, or 40 μg/kg initial dose and 0.5×IC₅₀, or 20 μg/kg, 2 weeks later; (2) 10×IC₅₀ initial dose and 5×IC₅₀ 2 weeks later; or 100×IC₅₀ and 50×IC₅₀ 2 weeks later.

TABLE 9
Examples of Clinical Cases

| | Status prior to | | |
|---|---|---|---|
| Patient No. | treatment | Treatment Regimen | Outcome |
| Patient 1 | No clinical signs, | 0.01-1 mg/kg | Maintenance and/or |
| | postmenopausal | biweekly for 4-24 | increase of muscle/bone |
| | and/or over 60 | weeks | mass |
| | years old | | |
| Patient 2 | Mild clinical signs, | 0.01-20 mg/kg | Maintenance and/or |
| | muscle wasting | weekly for 4 | increase of muscle/bone |
| | and/or bone loss | more weeks | mass |
| Patient 3 | Advanced stage of | 0.01-20 mg/kg | Improvement of clinical |
| | osteoporosis | twice weekly for | signs, maintenance and/or |
| | | 6 or more weeks | increase of muscle/bone |
| | | | mass |
| Patient 4 | Severe muscle | 0.01-20 mg/kg | Improvement of clinical |
| | and bone loss | daily for 6 or | signs, reduction in severity |
| | | more weeks | of symptoms and/or |
| | | | increase of muscle/bone |
| | | | mass |

### Example 12

### Treatment of Metabolic Disorders

Inhibitors of GDF-8, such as, for example, ActRIIB fusion polypeptides, are useful for treatment of metabolic disorders such as type 2 diabetes, impaired glucose tolerance, metabolic syndrome (e.g., syndrome X), insulin resistance induced by trauma (e.g., burns or nitrogen imbalance), and adipose tissue disorders (e.g., obesity). In the methods of the invention, the ActRIIB fusion polypeptides antibodies of the invention are used to treat a subject at disease onset or having an established metabolic disease.

Efficacy of ActRIIB fusion polypeptides for treatment of metabolic disorders, e.g., type 2 diabetes and/or obesity, is confirmed using well established murine models of obesity, insulin resistance and type 2 diabetes, including ob/ob, db/db, and strains carrying the lethal yellow mutation. Insulin resistance can also be induced by high fat or high caloric feeding of certain strains of mice, including C57BL/6J. Similarly to humans, these rodents develop insulin resistance, hyperinsuliemia, dyslipidemia, and deterioration of glucose homeostasis resulting in hyperglycemia. Outcome assessments are based on serum measurements of glucose, insulin and lipids. Measures of improved insulin sensitivity can be determined by insulin tolerance tests and glucose tolerance tests. More sensitive techniques would include the use of euglycemic-hyperinsulinemic clamps for assessing improvements is glycemic control and insulin sensitivity. In addition, the clamp techniques would allow a quantitative assessment of the role of the major glucose disposing tissues (muscle, adipose, and liver) in improved glycemic control.

In one study, treatment with an ActRIIB fusion polypeptide such one set out in SEQ ID NO:3 (IP injection) or vehicle is conducted for one week to six months. The treatment protocol could vary, with testing of different doses and treatment regimens (e.g., daily, weekly, or bi-weekly injections). It is anticipated that mice treated with the fusion polypeptide would have greater glucose uptake, increased glycolysis and glycogen synthesis, lower free fatty acids and triglycerides in the serum as compared to mice receiving placebo treatment.

The ActRIIB fusion polypeptides are also used to prevent and/or to reduce severity and/or the symptoms of the disease. It is anticipated that the ActRIIB fusion polypeptides would be administered as a subcutaneous injection as frequently as once per day and as infrequently as once per month. Treatment duration could range from one month and several years.

To test the clinical efficacy of ActRIIB fusion polypeptides in humans, subjects suffering from or at risk for type 2 diabetes are identified and randomized to a treatment group. Treatment groups include a placebo group and one to three groups receiving ActRIIB fusion polypeptides A different doses). Individuals are followed prospectively for one month to three years to assess changes in glucose metabolism. It is anticipated that individuals receiving treatment would exhibit an improvement.

The ActRIIB fusion polypeptides are administered as the sole active compound or in combination with another compound or composition. When administered as the sole active compound or in combination with another compound or composition, the dosage may be from approximately 1 μg/kg to 20 mg/kg, depending on the severity of the symptoms and the progression of the disease. The appropriate effective dose is selected by a treating clinician from the following ranges: 1 μg/kg to 20 mg/kg, 1 μg/kg to 10 mg/kg, 1 μg/kg to 1 mg/kg, 10 μg/kg to 1 mg/kg, 10 μg/kg to 100 μg/kg, 100 μg to 1 mg/kg, and 500 μg/kg to 1 mg/kg. Exemplary treatment regimens and outcomes are summarized in Table 7.

TABLE 7
Examples of Clinical Cases

| | Status prior to | Treatment | |
|---|---|---|---|
| Patient No. | treatment | Regimen | Outcome |
| Patient 1 | No clinical signs, | 0.01-1 mg/kg every 4 | Prevention of type 2 |
| | family history of | weeks for 48 weeks | diabetes |
| | type 2 diabetes | | |
| Patient 2 | Mild clinical signs | 0.01-20 mg/kg weekly | Improved insulin |
| | of syndrome X | for 4 more weeks | tolerance and glucose |
| | | | metabolism, and lower |
| | | | blood pressure |
| Patient 3 | Advanced stage of | 0.01-20 mg/kg twice | Improvement of clinical |
| | type 2 diabetes | weekly for 6 or more | signs, reduction in |
| | | weeks | severity of symptoms |
| | | | and/or increase in |
| | | | muscle mass/body fat |
| | | | ratio |
| Patient 4 | Severe insulin | 0.01-20 mg/kg daily for | Improvement of clinical |
| | resistance | 6 or more weeks | signs, reduction in |
| | and/obesity | | severity of symptoms |
| | | | and/or decrease in body |
| | | | fat |

1-28. (canceled)

29. A method for treating or preventing a muscle or neuromuscular disease or disorder that is associated with GDF-8 in a mammal comprising administering to the mammal an effective amount of a composition comprising an Activin Receptor Type IIB (ActRIIB) fusion polypeptide, wherein the fusion polypeptide comprises:
(a) an amino acid sequence chosen from:
(i) an amino acid sequence that is at least 80% identical to amino acids 19 to 134 of SEQ ID NO:1, or a fragment thereof that specifically binds GDF-8 or BMP-11; and
(ii) an amino acid sequence that is at least 80% identical to amino acids 23 to 138 of SEQ ID NO:3, or a fragment thereof that specifically binds GDF-8 or BMP-11; and
(b) an Fc portion of an antibody.

30. A method for treating or preventing a muscle or neuromuscular disease or disorder in a mammal in need thereof, comprising administering to the mammal an effective amount of a composition comprising an Activin Receptor Type IIB (ActRIIB) fusion polypeptide, wherein the fusion polypeptide comprises:
(a) an amino acid sequence encoded by a nucleic acid that hybridizes to the complement of nucleotides 67-414 of SEQ ID NO:4 under stringent hybridization conditions, or a fragment thereof that encodes a polypeptide that specifically binds GDF-8 or BMP-11; and
(b) an Fc portion of an antibody.

31. The method of claim 30, wherein the fusion polypeptide is encoded by a nucleic acid that hybridizes to the complement of SEQ ID NO:4.

32. The method of any of claims 29 to 31, wherein the muscle or neuromuscular disease or disorder is chosen from at least one of muscular dystrophy, Duchenne's muscular dystrophy, muscle atrophy, organ atrophy, carpal tunnel syndrome, congestive obstructive pulmonary disease, sarcopenia, frailty, cachexia, muscle wasting syndrome, damaged muscle, damaged myocardium, damaged diaphragm, glucocorticoid-induced muscle atrophy, and amyotrophic lateral sclerosis (ALS).

33. The method of claim 32, wherein the muscle or neuromuscular disease or disorder is Duchenne's muscular dystrophy.

34. The method of claim 32, wherein the muscle or neuromuscular disease or disorder is amyotrophic lateral sclerosis (ALS).

35. The method of claim 32, wherein the muscle or neuromuscular disease or disorder is sarcopenia.

36. A method of increasing muscle mass in a mammal in need thereof, comprising administering to the mammal an effective amount of a composition comprising an Activin Receptor Type IIB (ActRIIB) fusion polypeptide, wherein the fusion polypeptide comprises:
(a) an amino acid sequence chosen from:
(i) an amino acid sequence that is at least 80% identical to amino acids 19 to 134 of SEQ ID NO:1, or a fragment thereof that specifically binds GDF-8 or BMP-11; and
(ii) an amino acid sequence that is at least 80% identical to amino acids 23 to 138 of SEQ ID NO:3, or a fragment thereof that specifically binds GDF-8 or BMP-11;
and
(b) an Fc portion of an antibody.

37. A method of increasing muscle strength in a mammal in need thereof, comprising administering to the mammal an effective amount of a composition comprising an Activin Receptor Type IIB (ActRIIB) fusion polypeptide, wherein the fusion polypeptide comprises:
(a) an amino acid sequence chosen from:
(i) an amino acid sequence that is at least 80% identical to amino acids 19 to 134 of SEQ ID NO:1, or a fragment thereof that specifically binds GDF-8 or BMP-11; and
(ii) an amino acid sequence that is at least 80% identical to amino acids 23 to 138 of SEQ ID NO:3, or a fragment thereof that specifically binds GDF-8 or BMP-11;
and
(b) an Fc portion of an antibody.

38. A method of treating a metabolic disorder in a mammal in need thereof, comprising administering to the mammal an effective amount of a composition comprising an ActRIIB fusion polypeptide, wherein the fusion polypeptide comprises:
(a) an amino acid sequence chosen from:
(i) an amino acid sequence that is at least 80% identical to amino acids 19 to 134 of SEQ ID NO:1, or a fragment thereof that specifically binds GDF-8 or BMP-11; and
(ii) an amino acid sequence that is at least 80% identical to amino acids 23 to 138 of SEQ ID NO:3, or a fragment thereof that specifically binds GDF-8 or BMP-11;
and
(b) an Fc portion of an antibody.

39. The method of claim 38, wherein the metabolic disorder is one or more disorder chosen from type 2 diabetes, impaired glucose tolerance, metabolic syndrome, trauma-induced insulin resistance, and an adipose tissue disorder.

40. The method of claim 39, wherein the metabolic syndrome is syndrome X.

41. The method of claim 39, wherein the adipose tissue disorder is obesity.

42. A method of increasing glucose tolerance in a mammal in need thereof, comprising administering to the mammal an effective amount of a composition comprising an ActRIIB fusion polypeptide, wherein the fusion polypeptide comprises:
(a) an amino acid sequence chosen from:
(i) an amino acid sequence that is at least 80% identical to amino acids 19 to 134 of SEQ ID NO:1, or a fragment thereof that specifically binds GDF-8 or BMP-11; and
(ii) an amino acid sequence that is at least 80% identical to amino acids 23 to 138 of SEQ ID NO:3, or a fragment thereof that specifically binds GDF-8 or BMP-11;
and
(b) an Fc portion of an antibody.

43. The method of claim 29, wherein the mammal is human.

44. The method of claim 1, wherein the composition is a pharmaceutical composition.

45. The method of claim 29, wherein the amino acid sequence comprises at least 70 contiguous amino acids.

46. The method of claim 45, wherein the amino acid sequence comprises at least 80, 90, 100, 110 or 120 contiguous amino acids.

47. The method of claim 29, wherein the amino acid sequence is truncated.

48. The method of claim 29, wherein the ActRIIB fusion polypeptide comprises amino acids 23 to 138 of SEQ ID NO:3.

49. The method of claim 29, wherein the ActRIIB fusion polypeptide comprises amino acids 19 to 134 of SEQ ID NO:1.

50. The method of claim 29, wherein the ActRIIB fusion polypeptide comprises amino acids 23 to 119 of SEQ ID NO:3.

51. The method of claim 29, wherein the amino acid sequence is at least 85% identical to amino acids 23 to 138 of SEQ ID NO:3, or a fragment thereof that specifically binds to GDF-8 or BMP-11.

52. The method of claim 29, wherein the amino acid sequence is at least 90% identical to amino acids 23 to 138 of SEQ ID NO:3, or a fragment thereof that specifically binds to GDF-8 or BMP-11.

53. The method of claim 29, wherein the amino acid sequence is at least 80% identical to amino acids 23 to 119 of SEQ ID NO:3, or a fragment thereof that specifically binds to GDF-8 or BMP-11.

54. The method of claim 29, wherein the ActRIIB fusion polypeptide comprises an Fc portion of IgG.

55. The method of claim 54, wherein the Fc portion comprises amino acids 148 to 378 of SEQ ID NO:3.

56. The method of claim 29, wherein the ActRIIB fusion polypeptide comprises an Fc portion of IgG₁ or IgG₄.

57. The method of claim 29, wherein the ActRIIB fusion polypeptide comprises an antibody constant region.

58. The method of claim 29, wherein the Fc portion is modified to reduce effector function.

59. The method of claim 29, wherein the Fc portion is modified to reduce binding to an Fc receptor.

60. The method of claim 29, wherein the Fc portion is modified to reduce complement activation.

61. The method of claim 29, wherein the ActRIIB fusion polypeptide is glycosylated.

62. The method of claim 29, wherein the ActRIIB fusion polypeptide is pegylated.

63. The method of claim 29, wherein the ActRIIB fusion polypeptide is linked to a nonproteinaceous polymer.

64. The method of claim 63, wherein the nonproteinaceous polymer is chosen from polyethylene glycol, polypropylene glycol, and polyoxyalkylenes.

65. The method of claim 29, wherein the ActRIIB fusion polypeptide is chemically modified.

66. The method of claim 29, wherein the ActRIIB fusion polypeptide comprises a detectable label.

67. The method of claim 66, wherein the label is chosen from a radiolabel, an enzyme, and a chemical moiety.

68. The method of claim 29, wherein the circulatory half-life of the ActRIIB fusion polypeptide exceeds 5 days.

69. The method of claim 29, wherein the circulatory half-life of the ActRIIB fusion polypeptide exceeds 7 days.

70. The method of claim 29, wherein the circulatory half-life of the ActRIIB fusion polypeptide exceeds 10 days.

71. The method of claim 29, wherein the circulatory half-life of the ActRIIB fusion polypeptide exceeds 14 days.

72. The method of claim 29, wherein the ActRIIB fusion polypeptide specifically binds to GDF-8 with a Kₐ higher than 10⁶ M⁻¹.

73. The method of claim 29, wherein the ActRIIB fusion polypeptide specifically binds to GDF-8 with a Kₐ higher than 10⁸ M⁻¹.

74. The method of claim 29, wherein the ActRIIB fusion polypeptide specifically binds to GDF-8 with an ED₅₀ of 15 ng/ml.

75. The method of claim 29, wherein the ActRIIB fusion polypeptide specifically binds to BMP-11 with an ED₅₀ of 40 ng/ml.

76. The method of claim 29, wherein the ActRIIB fusion polypeptide has an IC₅₀ for inhibiting GDF-8 in the range of 0.07 nM to 0.1 nM.

77. The method of claim 29, wherein the ActRIIB fusion polypeptide is administered at an effective amount chosen from 1 μg/kg to 20 mg/kg, 1 μg/kg to 10 mg/kg, 1 μg/kg to 1 mg/kg, 10 μg/kg to 1 mg/kg, 10 μg/kg to 100 μg/kg, 100 μg to 1 mg/kg, and 500 μg/kg to 1 mg/kg.

78. The method of claim 29, wherein the ActRIIB fusion polypeptide is administered at an effective amount chosen from:
(1) a first dose at 1×IC₅₀, followed by a second dose at 0.5×IC₅₀, wherein the second dose is administered 2 weeks after the first dose;
(2) a first dose of 10×IC₅₀, followed by a second dose at 5×IC₅₀, wherein the second dose is administered 2 weeks after the first dose; and
(3) a first dose of 100×IC₅₀, followed by a second dose at 50×IC₅₀, wherein the second dose is administered 2 weeks after the first dose.


SEQUENCE LISTING (6 sequences)

SEQ ID NO: 1 - protein, 512 aa, Human
MTAPWVALALLWGSLCAGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 2 - protein, 375 aa, Human
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 3 - protein, 378 aa, Artificial Sequence
  Chimera/Fusion
MKFLVNVALVFMVVYISYIYATSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGRGDDDDKTRSRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPPK

SEQ ID NO: 4 - DNA, 1134 bp, Artificial Sequence
  Chimera/Fusion
atgaaattcttagtcaacgttgcccttgtttttatggtcgtgtacatttcttacatctatgcgactagtgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggcggccgcggagacgacgacgacaagacgcgttctagagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgcctaaa

SEQ ID NO: 5 - protein, 4 aa, Artificial Sequence
  Linking Sequence, Gly-Ser repeat
GSGS

SEQ ID NO: 6 - protein, 4 aa, Artificial Sequence
  Linking Sequence, Enterokinase Cleavage Site
DDDK